(19) United States
(12) Patent Application Publication
(10) Pub. No.: US 2012/0010502 A1
(43) Pub. Date: Jan. 12, 2012

(54) SYNCHRONIC MONITOR SYSTEM FOR DRUG DELIVERY INDUCED BY ULTRASOUND AND THE METHOD THEREOF

(75) Inventors: Feng-Yi YANG, Taipei (TW); Wei-Hsiu CHIU, Taipei (TW)

(73) Assignee: NATIONAL YANG MING UNIVERSITY, Taipei (TW)

(21) Appl. No.: 12/958,252

(22) Filed: Dec. 1, 2010

(30) Foreign Application Priority Data
Jul. 9, 2010 (TW) 099122710

(51) Int. Cl. A61N 7/00; A61B 8/06

(52) U.S. Cl. 600/431; 600/439

(57) ABSTRACT

The present invention provides a synchronic monitor system using real-time ultrasound image to monitor the permeable concentration of the targeted tissue for drug delivery induced by ultrasound. The drug delivery is performed by using a first ultrasound apparatus to emit a first ultrasound to a blood vessel of the targeted tissue to allow a drug passing through the blood vessel into a lesion zone. The synchronic monitor system comprises a second ultrasound apparatus and software. The second ultrasound apparatus has a transducer, and the transducer is used to emit a second ultrasound to the blood vessel to determine several information of the blood flow. The software is installed in the second ultrasound apparatus to collect the information of the blood flow, and calculate an extravasation of the drug in the lesion zone according to the information of the blood flow via a first equation. The present invention discloses a method using the same.

### CROSS-REFERENCE TO RELATED APPLICATIONS

This Non-provisional application claims priority under 35 U.S.C. §119(a) on Patent Application No(s). [099122710] filed on Jul. 9, 2010, in Taiwan, Republic of China, the entire contents of which are hereby incorporated by reference.

### BACKGROUND OF THE INVENTION

1. Field of the Invention

This invention relates to a monitor system for drug delivery induced by ultrasound and, more particularly, to a synchronic monitor system for monitoring an extravasation of a drug in a lesion zone and the method using the same.

2. Description of the Related Art

In present medical technology, delivering the drug to a lesion zone without passing the metabolism of the digestive system and the liver to maintain the concentration of the drug in the blood is a concerned research subject. However, it is difficult to deliver the drug to the lesion zone directly.

For example, the direct delivery of drugs to the central nervous system would make the resulting interactions highly target-specific and thereby dramatically improve the therapeutic effects abd reduce possible side effect. However, it is difficult to delivery many potent therapeutic agents to the brain due to the presence of the blood-brain barrier (BBB), which is a specialized system of capillary endothelial cells that protects the brain from harmful substances. Although many methods have been developed to overcome BBB impermeability when delivering drugs, such as increasing their liquid solubility, or by the using vectors such as amino acids for carriers, none has been applied clinically.

In recent year, focused ultrasound apparatus can be used to transiently disrupt the BBB and thereby aid the noninvasive delivery of treatment agents to specific regions in the brain. Before inducing the focused ultrasound apparatus, it is demonstrated that the administration of microbubbles as a ultrasound contrast agent can effectively enhance the efficiency of the drug delivery.

Please refer to FIG. 1, it is a diagram showing a drug extravasation as a function of the concentration of the ultrasound contrast agent according to the prior art. In FIG. 1, it shows that an extravasation of the drug is directly proportional to the concentration of the ultrasound contrast agent. That is, the extravasations of the drug will increase when the concentration of the ultrasound contrast agent increases.

In addition, numerous studies have focused on the use of contrast-specific medical imaging method to detect focused ultrasound enhanced drug delivery.

Although using the focus ultrasound apparatus can improve the efficiency of the drug delivery, it is still difficult to check whether the drug enters into the accurate targeted tissue or not, and further, it is difficult to check the concentration of the drug permeated into the targeted tissue.

Moreover, if we need to measure the concentration of the drug permeated from the blood vessel to the targeted tissue, we have to wait the finish of the sonication performed by the focused ultrasound apparatus and inject MRI contrast agent into the blood vessel for scanning. It is inconvenient and expensive, and can not get real-time feedback of the permeable status of the drug.

### BRIEF SUMMARY OF THE INVENTION

The present invention provides a synchronic monitor system using real-time ultrasound image to monitor the permeable concentration of the targeted tissue for drug delivery induced by ultrasound. The present invention can improve the efficiency of the drug in the lesion zone.

The synchronic monitor system in the present invention provides a synchronic monitor system using real-time ultrasound image to monitor the permeable concentration of the targeted tissue for drug delivery induced by ultrasound. The drug delivery is performed by using a first ultrasound apparatus to emit a first ultrasound to a blood vessel of the targeted tissue to allow a drug passing through the blood vessel into a lesion zone.

In one embodiment of the invention, the synchronic monitor system comprises a second ultrasound apparatus and software. The second ultrasound apparatus has a transducer, and the transducer is used to emit a second ultrasound to the blood vessel to determine several information of the blood flow. The software is installed in the second ultrasound apparatus to collect the information of the blood flow, and calculate an extravasation of the drug in the lesion zone according to the information of the blood flow via a first equation.

In one embodiment of the invention, the information of the blood flow comprises a mean blood flow velocity (MV), a peak systolic velocity (PSV) and a diastolic velocity (DV).

In one embodiment of the invention, the software calculates a pulsatility index (PI) according to the peak systolic velocity and the diastolic velocity via a second equation, the second equation is PI=(PSV−DV)/MV.

In one embodiment of the invention, the first equation is Y=−aX+bZ+C, where Y represents the extravasation of the drug in the lesion zone, X represents the normalized PSV change, and Z represents the normalized PI change.

In one embodiment of the invention, the normalized PSV change and the normalized PI change are obtained by using the second ultrasound apparatus to measure the PSV and the PI before and after sonication performed by the first ultrasound apparatus, and normalize the results corresponding to the measurement.

In one embodiment of the invention, the drug delivery further comprises an ultrasound contrast agent, the first ultrasound apparatus is performed along with the administration of the ultrasound contrast agent to the blood vessel.

In one embodiment of the invention, the concentration of the ultrasound contrast agent is directly proportional to the concentration of the drug permeated from the blood vessel to the targeted tissue, the normalized PSV change is inversely proportional to the concentration of the ultrasound contrast agent, and the normalized PI change is directly proportional to the concentration of the ultrasound contrast agent.

Another scope of the invention is to provide a synchronic monitor method. The synchronic monitor method provides drug delivery induced by ultrasound. The drug delivery is performed by using a first ultrasound apparatus to emit a first ultrasound to a blood vessel of the targeted tissue to allow a drug passing through the blood vessel into a lesion zone, the method comprises the following steps

In one embodiment of the invention, the method comprises the following steps: providing a second ultrasound apparatus including a transducer; emitting a second ultrasound by the second ultrasound apparatus to the blood vessel to determine several information of the blood flow; collecting the information of the blood flow, wherein the information of the blood flow comprising a mean blood flow velocity (MV), a peak systolic velocity (PSV) and a diastolic velocity (DV); calculating an extravasation of the drug in the lesion zone according to the information of the blood flow via a first equation; and calculating a pulsatility index (PI) according to the information of the blood flow via a second equation, wherein the second equation is PI=(PSV−DV)/MV.

These and other features, aspects, and advantages of the present invention will become better understood with regard to the following description, appended claims, and accompanying drawings.

### BRIEF DESCRIPTION OF THE DRAWINGS

FIG. 1 is a diagram showing a drug extravasation as a function of the concentration of the ultrasound contrast agent according to the prior art;

FIG. 2 is a schematic diagram showing a synchronic monitor system for drug delivery induced by ultrasound according to one embodiment of the invention;

FIG. 3A is a diagram showing a normalized PSV change as a function of the concentration of the ultrasound contrast agent according to one embodiment of the invention;

FIG. 3B is a diagram showing a normalized PI change as a function of the concentration of the ultrasound contrast agent according to one embodiment of the invention; and

FIG. 4 is a flow chart showing a synchronic monitor method for drug delivery induced by ultrasound according to one embodiment of the invention.

### DETAILED DESCRIPTION OF THE INVENTION

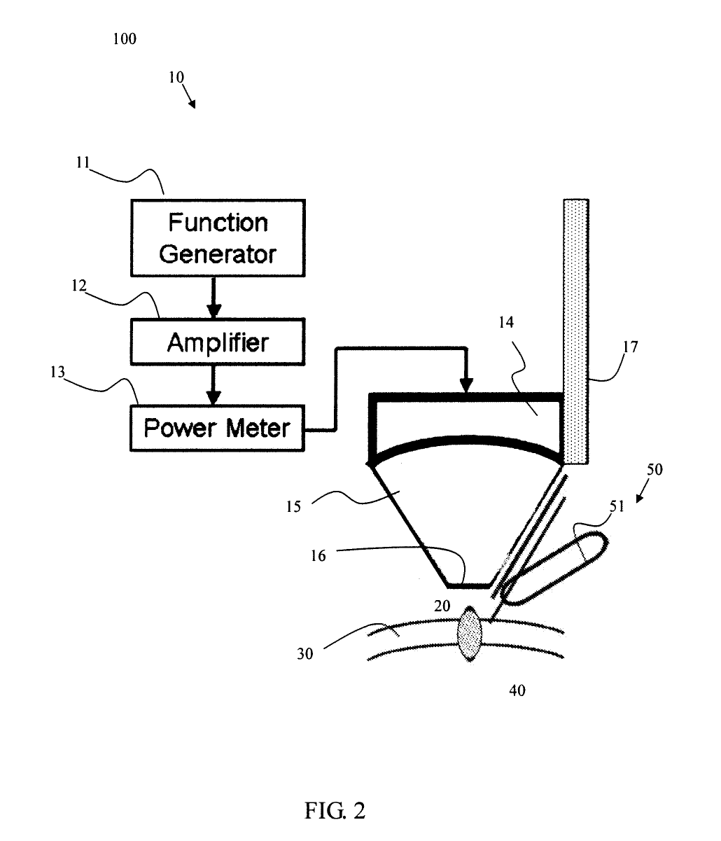

FIG. 2 is a schematic diagram showing an embodiment of a synchronic monitor system for drug delivery induced by ultrasound. The synchronic monitor system provides synchronic monitoring of drug delivery induced by ultrasound, wherein the drug delivery is performed by using a first ultrasound apparatus 10 to emit a first ultrasound to a blood vessel 30 of the targeted tissue 20 to allow a drug (not shown) passing through the blood vessel 30 into a lesion zone 40.

Please refer to FIG. 2, the synchronic monitor system comprises a second ultrasound apparatus 50 and software. The second ultrasound apparatus 50 has a second transducer 51, and the second transducer 51 is used to emit a second ultrasound to the blood vessel 30 to determine several information of the blood flow. The software is installed in the second ultrasound apparatus 50 to collect the information of the blood flow, and calculate an extravasation of the drug in the lesion zone 40 according to the information of the blood flow via a first equation.

In a preferred embodiment of the invention, the first ultrasound apparatus 10 is a Pulsed High-Intensity Focused Ultrasound with an aperture diameter of 64 mm, a radius of curvature of 62.64 mm, and a resonant frequency of 1 MHz. Moreover, the second ultrasound apparatus 50 is a Doppler ultrasound apparatus in the preferred embodiment, and a pulsed-wave Doppler ultrasound apparatus is more preferred. However, the aforementioned embodiments of the first ultrasound apparatus and the kind of the second ultrasound apparatus are for reference, and the invention is not limited thereto.

The information of the blood flow comprises a mean blood flow velocity (MV), a peak systolic velocity (PSV) and a diastolic velocity (DV).

According to FIG. 2, the first ultrasound apparatus 10 comprises a function generator 11, an amplifier 12, a power meter 13 and a first transducer 14. The first transducer 14 is mounted on a removable cone 15 filled with degassed water whose tips is sealed with a polyurethane membrane 16, and the center of the focal spot is positioned approximately 5 mm below the tip of the cone 15, however, the invention is not limited thereto. And then, the first transducer 14 is attached to a stereotaxic apparatus 17 that allowed 3-D positioning,

As shown in FIG. 2, the function generator 11 is connected to the power amplifier 12 to amplify the first ultrasound generated by the function generator 11, and the first ultrasound is then delivered to the first transducer via an electrical matching network. Finally, the first ultrasound apparatus emits the first ultrasound to the blood vessel 30 of the targeted tissue 20 to allow the drug passing through the blood vessel 30 into the lesion zone 40.

One embodiment used in the present invention is rat as following. First, the abdominal aortas of the rat is surgically exposed and sonicated with the first ultrasound apparatus 10 at a duty cycle of 5% and a pulse repetition frequency of 1 Hz. The total duration of the sonication is 6 s for each rat. However, the invention is not limited thereto. Various doses of the ultrasound contrast agents are then administered to the rats, for example, 0 μL/kg, 150 μL/kg, 300 μL/kg or 450 μL/kg. As to the efficacy of the ultrasound contrast agents has been described as the aforementioned, they are not described for a concise purpose. In the preferred embodiment, the ultrasound contrast agents are micro-bubbles.

Please refer to FIG. 2, the second ultrasound apparatus 50 is used for targeting whether the sonicated site performed by the first ultrasound apparatus 10 is correct, and synchronic monitoring the extravasation of the drug in the lesion zone. As the abovementioned, the second ultrasound apparatus 50 is a Doppler ultrasound apparatus, more particular, the second ultrasound apparatus 50 is a pulsed Doppler ultrasound apparatus used to produce a color Doppler image. In the invention, the angle between the blood vessel and the second ultrasound emitted by the second transducer 51 of the second ultrasound apparatus 50 does not exceed 60°.

The second ultrasound apparatus 50 can measure MV₀, PSV₀ and DV₀ of the blood in the blood vessel 30, wherein MV₀, PSV₀ and DV₀ are the value of the MV, the PSV and the DV for the various doses of the ultrasound contrast agents before sonication performed by the first ultrasound apparatus 10, respectively. The MV, the PSV and the DV of the blood flow in the blood vessel 30 are then measured immediately after the second transducer 51 emits the second ultrasound to the blood vessel 30.

After obtaining MV₀, MV, PSV₀, PSV, DV₀ and DV, the software installing in the second ultrasound apparatus 50 can calculate PI₀ according to MV₀, PSV₀ and DV₀ via a second equation PI₀=(PSV₀−DV₀)/MV₀, wherein PI₀ is the value of the PI for the various doses of the ultrasound contrast agents before sonication performed by the first ultrasound apparatus 10. The PI can also obtain according to MV, PSV and DV via the second equation.

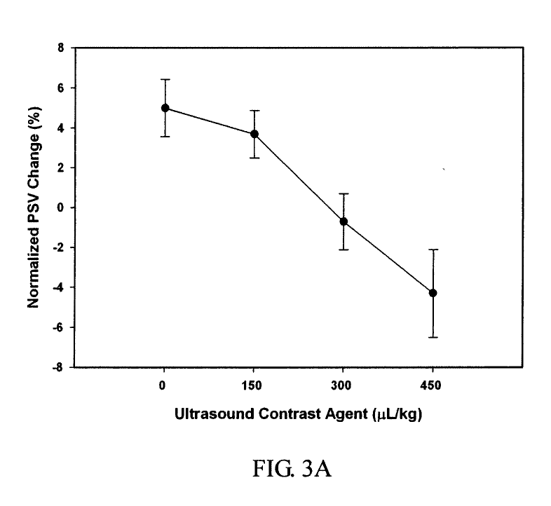

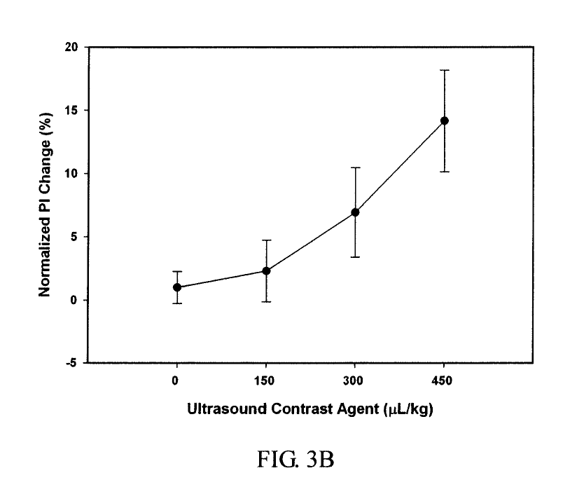

Then, the software normalizes PSV₀ and PSV measured by the second ultrasound apparatus 50, that is, the software calculates the normalized PSV change according to PSV₀ and PSV via an equation (PSV-PSV_{O})/PSV₀. FIG. 3A is a diagram showing a normalized PSV change as a function of the concentration of the ultrasound contrast agent according to one embodiment of the invention. All PSV changes are inversely proportional to the concentration of the ultrasound contrast agent as shown in FIG. 3A.

The software also normalizes PI₀ and PI, that is, the software calculates the normalized PI change according to PI₀ and PI via an equation (PI−PI₀)/PI₀ FIG. 3B is a diagram showing a normalized PI change as a function of the concentration of the ultrasound contrast agent according to one embodiment of the invention. All PI changes are directly proportional to the concentration of the ultrasound contrast agent as shown in FIG. 3B.

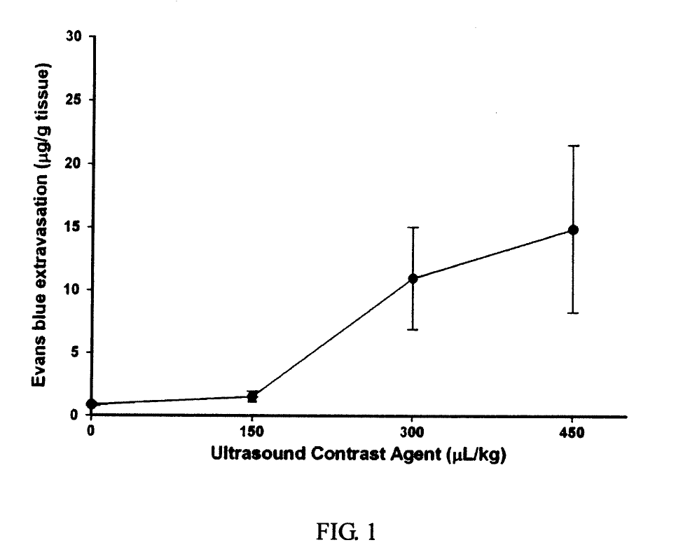

Please refer to FIG. 1, FIG. 3A and FIG. 3B, the relationships between the concentration of the ultrasound contrast agent, the PSV change and the PI change can be measured, calculate and obtained. Therefore, the extravasation of the drug can be calculated according a first equation Y=−aX+bZ+C, wherein Y represents the extravasation of the drug in the lesion zone, X represents the normalized PSV change, and Z represents the normalized PI change.

According to the synchronic monitor system provided in the present invention, the permeability of the drug in the lesion zone can be real-time monitored because the second ultrasound apparatus 50 can measure the information of the blood flow in the blood vessel 30 synchronously while the blood vessel is exposed and sonicated with the first ultrasound apparatus 10.

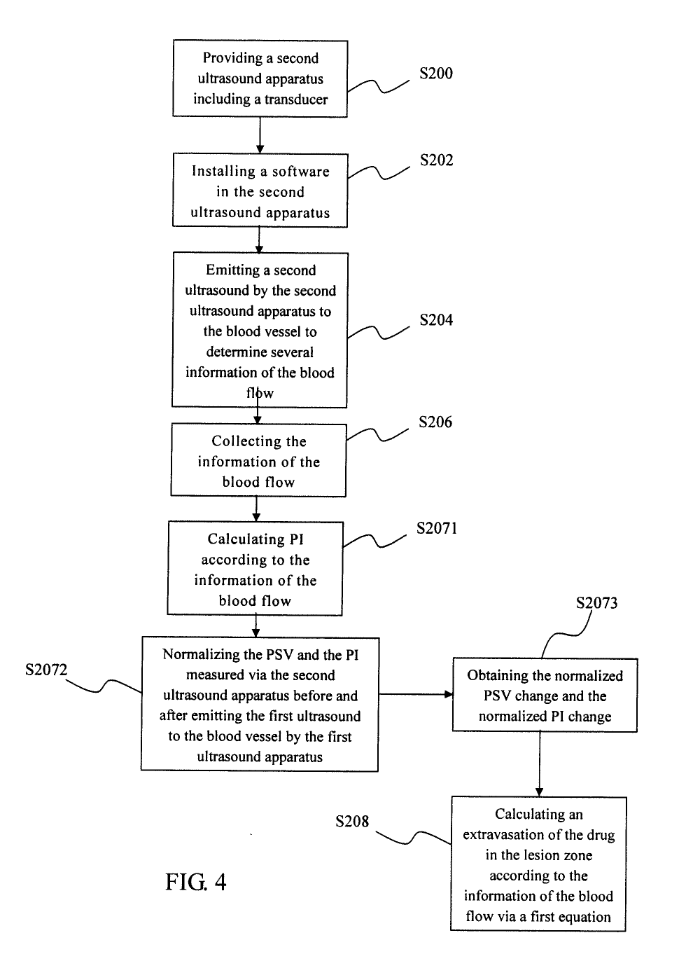

FIG. 4 shows a flow chart of a synchronic monitor method for drug delivery induced by ultrasound according to one embodiment of the invention. The synchronic monitor method starts by step S200 of providing a second ultrasound apparatus including a transducer. Step S200 is followed by step S202 of installing a software into the second ultrasound apparatus. Step S202 is followed by step S204 of emitting a second ultrasound by the second ultrasound apparatus to the blood vessel to determine several information of the blood flow. Step S206 is performed by the software to collect the information of the blood flow, wherein the information of the blood flow comprises a mean blood flow velocity (MV), a peak systolic velocity (PSV) and a diastolic velocity (DV). Step S206 is followed by step S208 of calculating an extravasation of the drug in the lesion zone according to the information of the blood flow via a first equation.

The synchronic monitor method in the present invention further comprises the following steps: Step S2071 is performed by the software to calculate a pulsatility index (PI) according to the information of the blood flow via a second equation, wherein the second equation is PI=(PSV−DV)/MV.

In addition, step S204 of emitting a second ultrasound by the second ultrasound apparatus to the blood vessel to determine several information of the blood flow can be performed repeatedly before and after the first ultrasound apparatus emits the first ultrasound to the blood vessel for obtaining PSV₀, PI₀, PSV and PI, respectively. Therefore, the synchronic monitor method in the present invention further comprises the following step: step S2072 is performed by normalizing PSV₀, PI₀, PSV and PI. Step S2072 is followed by step S2073 of obtaining the normalized PSV change and the normalized PI change.

Furthermore, the extravasation of the drug can be calculated by the software according the normalized PSV change and the normalized PI change via a first equation Y=−aX+bZ+C, wherein Y represents the extravasation of the drug in the lesion zone, X represents the normalized PSV change, and Z represents the normalized PI change; and further, coefficient a, b and constant C can be obtained using various values of the extravasations of the drug, the normalized PSV changes and the normalized PI changes in the first equation.

Finally, the method in the present invention further comprises a step of administering a ultrasound contrast agent to the blood vessel (not shown). As to the efficacy of the ultrasound contrast agents has been described as the aforementioned, they are not described for a concise purpose.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A synchronic monitor system for drug delivery induced by ultrasound, the drug delivery is performed by using a first ultrasound apparatus to emit a first ultrasound to a blood vessel of the targeted tissue to allow a drug passing through the blood vessel into a lesion zone, the synchronic monitor system comprising:
a second ultrasound apparatus including a transducer, the transducer used to emit a second ultrasound to the blood vessel to determine several information of the blood flow; and
software installing in the second ultrasound apparatus to collect the information of the blood flow, and calculate an extravasation of the drug in the lesion zone according to the information of the blood flow via a first equation.

2. The synchronic monitor system according to claim 1, wherein the information of the blood flow comprises a mean blood flow velocity (MV), a peak systolic velocity (PSV) and a diastolic velocity (DV).

3. The synchronic monitor system according to claim 2, wherein the software calculates a pulsatility index (PI) according to the peak systolic velocity and the diastolic velocity via a second equation, the second equation is PI=(PSV−DV)/MV.

4. The synchronic monitor system according to claim 3, wherein the first equation is Y=−aX+bZ+C, where Y represents the extravasation of the drug in the lesion zone, X represents the normalized PSV change, and Z represents the normalized PI change.

5. The synchronic monitor system according to claim 4, wherein the normalized PSV change and the normalized PI change are obtained by using the second ultrasound apparatus to measure the PSV and the PI before and after sonication performed by the first ultrasound apparatus, and normalize the results corresponding to the measurement.

6. The synchronic monitor system according to claim 1, wherein the drug delivery further comprises an ultrasound contrast agent, the first ultrasound apparatus is performed along with the administration of the ultrasound contrast agent to the vessel.

7. The synchronic monitor system according to claim 6, wherein the concentration of the ultrasound contrast agent is directly proportional to the concentration of the drug permeated from the blood vessel to the targeted tissue, the normalized PSV change is inversely proportional to the concentration of the ultrasound contrast agent, and the normalized PI change is directly proportional to the concentration of the ultrasound contrast agent.

8. The synchronic monitor system according to claim 1, wherein the first ultrasound apparatus is a pulsed high-intensity focused ultrasound apparatus, the second ultrasound apparatus is a Doppler ultrasound apparatus.

9. A synchronic monitor method for drug delivery induced by ultrasound, the drug delivery is performed by using a first ultrasound apparatus to emit a first ultrasound to a blood vessel of the targeted tissue to allow a drug passing through the blood vessel into a lesion zone, the method comprises the following steps:
providing a second ultrasound apparatus including a transducer;
emitting a second ultrasound by the second ultrasound apparatus to the blood vessel to determine several information of the blood flow;
collecting the information of the blood flow, wherein the information of the blood flow further comprising a mean blood flow velocity (MV), a peak systolic velocity (PSV) and a diastolic velocity (DV);
calculating an extravasation of the drug in the lesion zone according to the information of the blood flow via a first equation; and
calculating a pulsatility index (PI) according to the information of the blood flow via a second equation, wherein the second equation is PI=(PSV−DV)/MV.

10. The synchronic monitor method according to claim 9, wherein the first equation is
Y=−aX+bZ+C, where Y represents the extravasation of the drug in the lesion zone, X represents the normalized PSV change, and Z represents the normalized PI change.

12. The synchronic monitor method according to claim 9, wherein the step of emitting a second ultrasound by the second ultrasound apparatus to the blood vessel to determine several information of the blood flow can be performed repeatedly before and after the step of emitting the first ultrasound to the blood vessel of the targeted tissue by the first ultrasound apparatus.

13. The synchronic monitor according to claim 9, the method further comprises the following steps:
normalizing the PSV and the PI measured via the second ultrasound apparatus before and after emitting the first ultrasound to the blood vessel by the first ultrasound apparatus; and
obtaining the normalized PSV change and the normalized PI change.

14. The synchronic monitor method according to claim 9, the method further comprises the following step:
administering a ultrasound contrast agent to the vessel.
